(19) United States
(12) Patent Application Publication
(10) Pub. No.: US 2024/0009439 A1
(43) Pub. Date: Jan. 11, 2024

(54) SUBDURAL EVACUATION PORT WITH NEEDLE ACCESS PORT

(71) Applicant: Asfora IP, LLC, Sioux Falls, SD (US)

(72) Inventor: Wilson T. Asfora, Sioux Falls, SD (US)

(21) Appl. No.: 18/347,474

(22) Filed: Jul. 5, 2023

Related U.S. Application Data
(60) Provisional application No. 63/481,094, filed on Jan. 23, 2023.
(60) Provisional application No. 63/367,719, filed on Jul. 5, 2022.

(51) Int. Cl. A61M 39/02; A61M 39/04

(52) U.S. Cl. CPC A61M 39/0247; A61M 39/04; A61M 2039/0276; A61M 2039/025

(57) ABSTRACT

The present disclosure relates to a subdural evacuation port device for evacuating the subdural space of a patient. The device includes a body that includes: a distal opening at a distal end, a primary evacuation opening at a proximal end, a skull engagement region that at least partially surrounds the distal end of the body, and a primary lumen extending from the distal opening to the primary evacuation opening. The skull engagement regions is also configured to engage with a skull of a patient. The body is formed from a polymer that is sufficiently rigid to sustain suction in the lumen to allow withdrawal of subdural fluid from the distal opening through the primary lumen to a suction device connected to the primary evacuation opening.

### PRIORITY

This application claims priority to U.S. Provisional Appl. No. 63/367,719 filed on Jul. 5, 2022. This application also claims priority to U.S. Provisional Appl. No. 63/481,094 filed on Jan. 23, 2023. The disclosure of each of these priority applications is incorporated herein by reference in its entirety.

### FIELD OF DISCLOSURE

The present disclosure relates to systems for removing fluids from the subdural region of a patient and more particularly pertains to a subdural evacuating port system for removing subdural fluid accumulations in a manner that is minimally invasive and promotes decompression, expansion, and recovery of the brain.

### BACKGROUND

The subdural space of the human head is the space located between the brain and the lining of the brain, which is referred to as the dura mater (hereinafter referred to as the “dura”). Hemorrhages on the surface of the brain, for example, may cause a condition known as a subdural hematoma. The subdural hemorrhages may have a number of causes. For example, elderly persons may be more susceptible to subdural hemorrhages because as the brain ages it tends to become atrophic and the subdural space between the brain and the dura gradually enlarges. Bridging veins between brain and dura frequently stretch and rupture as a consequence of relatively minor head injuries, thus giving rise to a collection of blood in the subdural space. Further, severe linear acceleration or deceleration of the brain can result in the brain moving excessively with respect to the dura, often causing rupture of the bridging veins or the blood vessels on the surface of the brain, which can in turn cause subdural hemorrhages in an otherwise healthy brain.

These subdural blood collections can be classified as acute subdural hematomas, subacute subdural hematomas, and chronic subdural hematomas. Acute subdural hematomas, which are associated with major cerebral trauma, generally consist primarily of fresh blood. Subacute subdural hematomas are generally associated with less severe injuries than those underlying the acute subdural hematomas. Chronic subdural hematomas are generally associated with even less severe, or relatively minor, injuries. The chronic subdural hematomas tend to be less dense liquid consisting of very diluted blood. Another condition involving a subdural collection of fluid is a hygroma, which is a collection of cerebrospinal fluid (sometimes mixed with blood) beneath the dura, which may be encapsulated.

While devices for evacuation of fluids form the subdural space are known, such devices may not be able to remove the fluids when a blockage (e.g., blood clots) is encountered within the subdural space and/or enters the evacuation device (e.g., during aspiration the subdural fluid by suctioning).

### SUMMARY

The present disclosure relates to systems, methods and devices for evacuating fluid from the subdural space of a patient.

In some aspects, some of the disclosed devices relate to a subdural evacuation port device that may include a distal opening at a distal end, a primary evacuation opening at a proximal end, a skull engagement region that at least partially surrounds the distal end of the body, and a primary lumen extending from the distal opening to the primary evacuation opening. The skull engagement region may be configured to engage with a skull of a patient. The body may be formed from a polymer that is sufficiently rigid to sustain a suction in the lumen to allow withdrawal of subdural fluid from the distal opening through the lumen to a suction device connected to the primary evacuation opening.

In various embodiments, the polymer can have a substantially low thrombogenicity. Optionally, the device can be formed from polyacetal. The subdural evacuation port device can also be made from a material that is compatible for use during computerized tomography (CT) scans.

In various embodiments, the subdural evacuation port device can further include a needle access port that defines an access port lumen in communication with the primary lumen, and a seal at a proximal end of the access port lumen that seals the access port lumen to preserve the suction within the lumen and that is penetrable by a needle.

In various embodiments, the needle access port can be disposed and oriented with respect to the body to allow a needle to be inserted into the access port to extend into the primary lumen.

Optionally, the polymer of the needle access port can be optically transparent.

In various embodiments, the skull engagement region can include threads for cutting threads into the skull of the patient. The threads can be fluted and self-tapping.

In another aspect, the present disclosure relates to a subdural evacuation port device that includes a body. The body may include: a distal opening, a primary evacuation opening, and a primary lumen extending from the distal opening to the primary evacuation opening. The subdural evacuation port device may additionally include a needle access port defining an access port lumen that is in communication with the primary lumen. The needle access port may be positioned with respect to the body to direct a needle inserted within the access port lumen into the primary lumen. Optionally, the body can be formed from a polymer.

In various embodiments, the needle access port can include a stop surface positioned at a first distance away from the distal opening to stop insertion of the needle past a threshold depth of insertion into the access port lumen.

In various embodiments, the primary evacuation opening can be configured to connect to an evacuation device that is configured to provide suction through the primary lumen.

In some embodiments, the subdural evacuation port device can optionally include a seal that seals the access port lumen to preserve suction within the primary lumen and that is penetrable by the needle.

Optionally, the subdural evacuation port device can include a second needle access port defining a second access port lumen in communication with the primary lumen. The second needle access port may be configured to direct a needle inserted within the second access port lumen and into the primary lumen, and at a location or an orientation that is different from that of a needle inserted within the access port lumen.

In some embodiments, the subdural evacuation port device can be compatible for use during computerized tomography (CT) scans.

At Least Certain Portions of the Subdural Evacuation Port Device May Optionally be Formed from an Optically Transparent Material.

In some embodiments, the subdural evacuation port device can be used and/or included in a kit for evacuating fluid from subdural space of a skull. The kit can include a needle configured for insertion into the port lumen for performing and operation at a blockage site for removal of a blockage within the primary lumen.

In various embodiments, the needle access port of the subdural evacuation port device can include a stop surface positioned at a first distance away from the distal opening to stop insertion of the needle past a threshold depth of insertion into the access port lumen.

Optionally, the needle can be a lumbar puncture needle.

The kit can additionally include an evacuation device that is configured to provide suction through the primary lumen. The evacuation device can be a bulb pump.

In some embodiments, the needle of the kit can be hollow and includes a needle lumen. The kit can additionally include a stylet configured to removably extend through the needle lumen to puncture a seal that seals the access port lumen of the subdural evacuation port device while preserving suction within the primary lumen.

In another aspect, the subdural evacuation port can be used in performing a method for removing fluid from the subdural space of a patient. The method can include drilling a hole through the skull and dura of a patient, mounting the subdural evacuation port device to the hole via a skull engagement region surrounding the distal end of the body, applying suction within the primary lumen to withdraw subdural fluid from the dura of the patient through the primary lumen, determining whether there is a blockage preventing subdural fluid from flowing through the primary lumen and in response to determining that a blockage is preventing subdural fluid from flowing through the primary lumen, removing the blockage.

In some embodiments, determining the cause of the blockage can be completed by performing a CT scan.

The method can additionally include removing the blockage by inserting a hollow needle through the access port lumen into the primary lumen. The method can additionally include injecting a thrombolytic agent via a hollow needle lumen of the needle The method can further include suctioning the blockage using the needle.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1 is a side view of an embodiment of a subdural evacuation port device;

FIG. 2 is a perspective view of the port device of FIG. 1 being used to extract subdural fluid;

FIG. 3 is a side cross-sectional view of the system shown in FIG. 2;

FIG. 4 is a side view of an example needle and stylet for use with the embodiments of the evacuation port device;

FIG. 5 is a perspective view of an example retractor as shown in FIGS. 2 and 3;

FIG. 6 is a cutaway side-view of another embodiment of a subdural evacuation port device;

FIG. 7 is a side view of another embodiment of a subdural evacuation port device;

FIG. 8 is a side/proximal perspective view of a subdural evacuation port device of FIG. 7;

FIG. 9 is a distal view of another embodiment of a subdural evacuation port device of FIG. 7; and

FIG. 10 is flow chart showing an example of a method of using a subdural evacuation port device in accordance with the principles of the present disclosure.

### DETAILED DESCRIPTION

The systems and methods will now be described hereinafter with reference to the accompanying drawings and examples, in which various embodiments are shown. However, the disclosure is now so limiting, and me be embodied in many different forms and should not be construed as limited to the embodiments set forth herein. Rather, these embodiments are provided so that this disclosure will be thorough and complete, and will fully convey the scope of the invention to those skilled in the art.

As discussed above, devices for clearing the subdural space of the human brain have been used successfully (for example, see U.S. Pat. No. 7,694,821). However, in some cases, these devices can become blocked, for example, by a blood clot. Such blockages can lead to slowing or stopping of the evacuation of fluid from the subdural space. Furthermore, the blockage can cause creation of high or uneven pressure within the evacuation port device and/or the subdural space potentially causing or increasing the risk of brain damage and hampering the recovery of brain. Clearing the blockage can require removal and reinsertion the of the device and present the risk of additional damage or hemorrhaging of the patient's brain. The present disclosure describes devices and methods that address at least some of the issues described above and/or other issues.

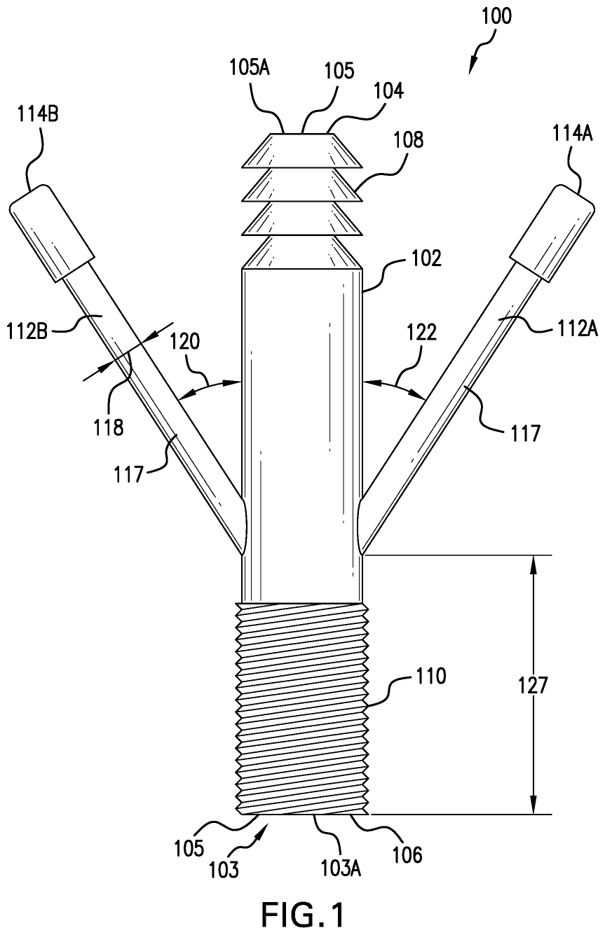

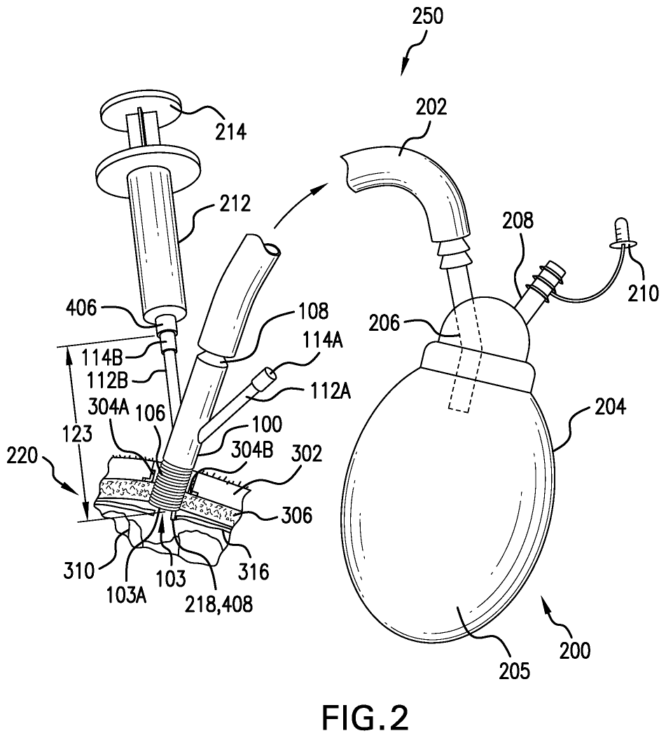

Referring to FIGS. 1-3, an exemplary subdural evacuation port device 100 is described. Subdural evacuation port device 100 includes a body 102 with a proximal end 104 and a distal end 106. The body 102 includes a primary lumen 103 extending from the distal end 106 to the proximal end 104 defining a fluid passage between the distal end 106 and the proximal end 104. The proximal end 104 of the body 102 includes an evacuation portion 105 including a primary evacuation opening 105A that is configured to attach to an evacuation device (for example, via a fitting 108) to evacuate or aspirate fluid from the subdural space, via the primary lumen 103. For example, the evacuation portion 105 can include a fitting 108 on the outside surface that is configured to operably connect to the evacuation device. In some embodiments, the fitting 108 is connected to a conduit 202 (shown in FIG. 2) which is in turn connected to the evacuation device 200. In the figures, the fitting 108 is a barbed tube fitting, however the disclosure is not so limiting. Other fittings are additionally possible, for example, the fitting 108 can be a female fitting, a threaded tube, pipe fitting, friction fitting, or other suitable fitting to attach to an evacuation device.

As discussed below, the body 102 is made from a material that is sufficiently rigid to prevent collapse of the body and subsequent closure of primary lumen 103 when suction (e.g., via negative pressure, partial vacuum, etc.) is applied to subdural evacuation port device 100 by an evacuation device (e.g., device 200 shown in FIG. 2).

The primary lumen 103 includes a distal opening 103A that is configured to be inserted within the subdural space (using any now or hereafter known methods), and for draining fluid therefrom. A diameter of the primary lumen 103 is configured to be sufficiently wide to drain fluid without excessive pressure on the subdural space or skull of a patient. The diameter of the primary lumen 103 may be constant between the proximal end 104 and the distal end 106, and/or optionally may change between the proximal end 104 and the distal end 106.

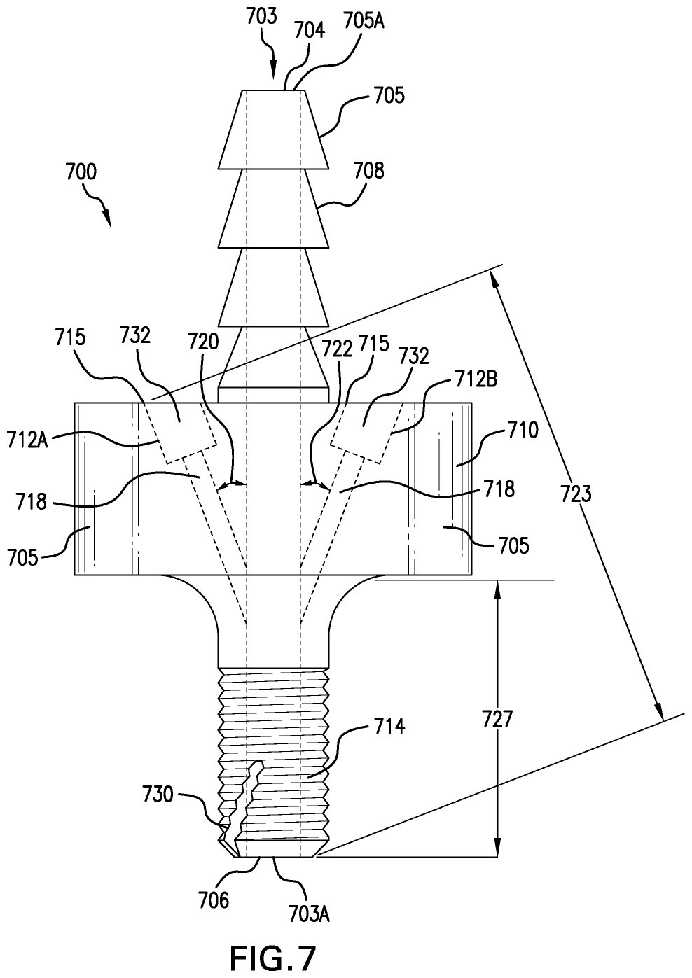

The distal end 106 includes a skull engagement region 110 to engage with the skull of a patient. In some embodiments, the skull engagement region 110 includes external threads to engage with the skull of a patient. Other mechanisms allowing for engagement with a skull are additionally possible such as, without limitation, friction fit, drilling, tapping, or a similar engagement mechanism. In some other embodiments, the subdural evacuation port device 100 includes threads as the skull engagement region 110 that are fluted and/or are self-tapping. Self-tapping threads (e.g., shown in FIG. 7) can tap a hole as subdural evacuation port device 100 is driven into the skull of the patient. In some embodiments, the threads 110 are hexagonal threads. In some other embodiments, the threads 110 are pipe threads (e.g., male pipe threads [MPT], female pipe threads [FPT], national pipe threads [NPT] or Whitworth threads). Pipe threads can be configured such that the skull engagement region is angled towards the primary lumen 103 while the primary lumen 103 remains constant. For example, when using pipe threads, a proximal side of the threads can have a larger diameter than the distal side of the threads, thereby enhancing bone purchase when engaged with a skull. In a preferred embodiment, the proximal side of the threads have a diameter of about 7 mm, 7.5 mm, about 8 mm or any other suitable diameter and the distal side of the threads have a diameter of about 4.5 mm, 5 mm, about 5.5 mm, about 6 mm, or any other suitable diameter. In some embodiments, the proximal side of the threads have a diameter between about 7-8 mm, about 7.2-7.8 mm, about 7.4-7.6 mm or any other suitable range of diameters and the distal side of the threads have a diameter between, 4-6 mm about 4.5 mm-5.5 mm, about 4.7-5.2 mm, or any other suitable range of diameters. Other suitable embodiments are additionally possible. In some other embodiments, the threads are configured as straight threads and not include an angle. In some other embodiments, the threads 110 are trapezoidal threads. In some examples, the threads are coarse and have a pitch of about 1 mm, about 1.5 mm, about 2 mm, about 2.5 mm or any other suitable pitch. In some examples, the threads have a pitch between about 1 mm-2.5 mm, about 1.2 mm-2.2 mm, about 1.4 mm-2 mm or any other suitable range of diameters.

Subdural evacuation port device 100 also includes needle access ports 112A, 112B including access port lumens 118 which are in fluid communication with the primary lumen 103 and configured to assist in removal of blockages from the distal opening 103A, from proximity of the distal opening in the subdural space, and/or other portions of the primary lumen 103. For example, a blockage situated in proximity of (within the subdural space) and/or partially within the distal opening 103A may reduce and/or prevent suction of fluids from the subdural device, via the primary lumen 103. Such blockages may be removed, broken, repositioned, and/or otherwise operated on using a tool inserted via one or more of the needle access ports 112A, 112B.

In various embodiments, the needle access ports allow a user to perform an operation—using a tool—at the site of a blockage (e.g, within the blockage and areas surrounding the blockage) to assist in removal of the blockage without interfering with the fluid evacuation via the primary lumen and without requiring removal/reinsertion of the evacuation port device. Specifically, the access port lumens 118 can receive a tool inserted through the needle access ports 112A, 112B and exit the distal opening 103A of the primary lumen 103 to access an area in the subdural space near the distal opening 103A (and/or access an area of the primary lumen 103 near the distal opening 103A). Examples of the operation may include, without limitation, causing breakage of the blockage, removal of the blockage (or debris/pieces thereof) by suction or aspiration, repositioning of the blockage (or pieces thereof), injection of a thrombolytic agent at the site of a blockage, or the like. Furthermore, a length of a tool used for performing the operation via the needle access port can be controlled (discussed below) such that it does not extend deeply into the subdural space of the human brain or contact soft tissue, thereby allowing the user to perform the operation for assisting in the removal of blockages without causing damage.

While the figures show two needle access ports, the disclosure is not so limiting and any number of needle access ports may be present (e.g., 1, 2, 3, 4 . . . ). In some embodiments, multiple needle access ports may provide multiple different points to reach the blockage site for performing an operation. As discussed above, each needle access port includes access port lumens 118 that is sized to receive a tool (e.g., needle) and guide it into the primary lumen 103, to exit out of the distal opening 103A. In some embodiments, the port lumens 118 can be about 1 mm, about 2 mm, about 3 mm, about 4 mm or any other suitable diameter. In some examples, the access port lumens can be between 1 mm-4 mm, about 1.5-2.5 mm, about 1.8 mm-2.3 mm or any other suitable range of diameters. In other embodiments, the access port lumens can be sized to receive other suitable tools. The diameters and lengths of the needle access ports 112A and 112B are configured to reliably position the tip of an inserted tool (e.g., a needle) at or near the site of blockage such as into the subdural space and at or near the distal opening 103A of the primary lumen 103. In some embodiments, access port lumen 118 is tapered to provide further guidance to the tool and allows for the tool to extend completely through the port lumen 118. In some other embodiments, the port lumen 118 has a uniform diameter that is larger than the diameter of the needle that is inserted. In some other embodiments, the access port lumen 118 has a non-uniform diameter with at least a minimum diameter which is larger than the diameter of a needle.

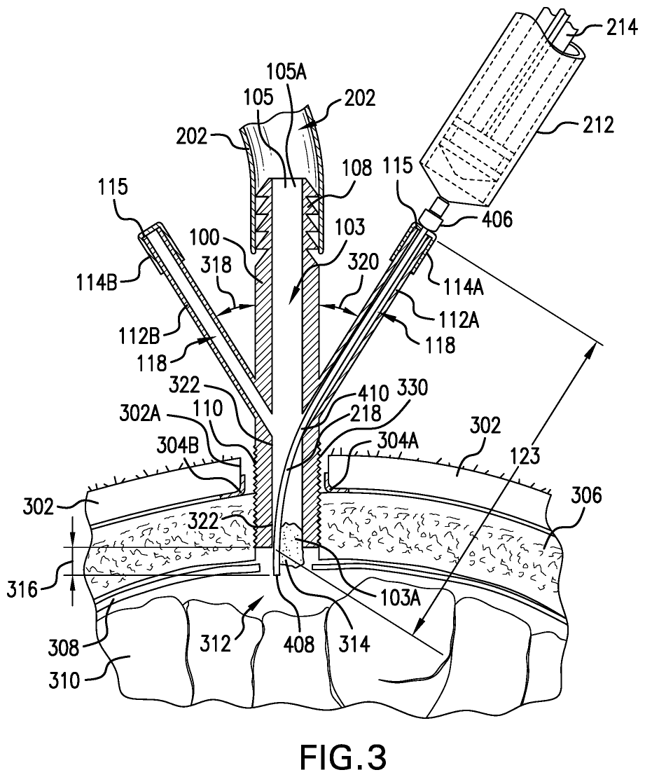

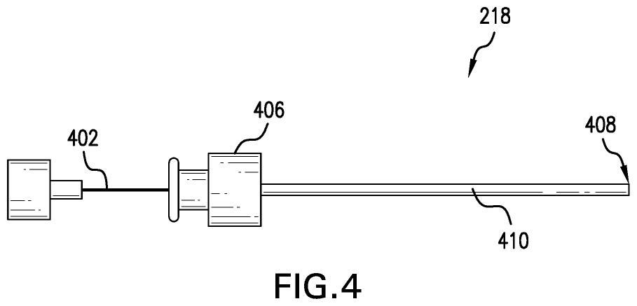

The needle access ports 112A, 112B are disposed at angles 120, 122 respectively with respect to the body 102. In some embodiments, angles 120, 122 provide guidance for a tool such as a needle (e.g., needle 218 shown in FIGS. 3 and 4) to be inserted into the side port to reach the distal opening 103A of body 102. In other examples, the angles 120, 122 can guide the needle to the interior side wall 322 of body 102 (e.g., see FIG. 3). In the case where the needle access ports guide the needle into an interior side wall 322, the tool or needle can be configured to flex and be directed towards distal opening 103A. In some embodiments, the angles 120, 122 may be the same or different. In some embodiments, angles 120, 122 are up to about 50 degrees. In further embodiments, angles 120, 122 are angles up to about 40 degrees. In further embodiments, angles 120, 122 are angles up to about 30 degrees. In some embodiments, angles 120, 122 are at least about 15 degrees. In some embodiments, needle access ports 112A, 112B can be located on opposite sides of body 102 (i.e., 180 degrees apart). In other embodiments, needle access ports 112A, 112B can be located in other locations radially around the primary lumen 103 to provide multiple access points to the blockage. For example, if one withdrawal mechanism does not hit the blockage or sufficiently clear the blockage, another withdrawal mechanism can be inserted in a different position. Thus, needle access ports may be placed at different heights and/or radial locations about the body 102 and at different angles with respect to the body 102.

In some embodiments, the needle access ports 112A, 112B can be defined by rigid protrusions 117 extending from body 102. Optionally, the needle access ports may be semi-rigid or flexible protrusions extending form the body 102. In other embodiments, needle access ports can be integral with the body (e.g., see FIG. 7). Other needle access ports allowing for fluid communication with the primary lumen 103 are additionally possible.

In some examples, the tool is a needle such as a flexible needle (e.g., see FIG. 4). Optionally, a syringe may be provided at the proximal end of the flexible needle for, for example, deliver a thrombolytic agent via the needle, aspirating the blockage (or pieces thereof), or the like. Optionally, the needle can be a lumbar puncture (“LP”) needle. Other suitable tools are additionally possible such as a tool which is configured to provide suction pressure (or aspiration), delivery of a thrombolytic agent, a reposition the blockage, and/or break-up a blockage is within the scope of this disclosure. In certain embodiments, the needle can be rigid or flexible. For example, if the needle is flexible, the needle can bend around the inner wall of body 102 to reach the distal end 106 of the body 102.

The needle can vary in size based on the dimensions of the subdural evacuation port device, or vice-versa. For example, the needle can be an 18-gauge LP needle. The needle can additionally have a different suitable gauge for example, a 16-gauge needle, a 19-gauge need, a 20-gauge needle, a 21-gauge or any other suitable gauge of needle. The needle can be of various lengths in some embodiments the needle can have a length of about 5 cm, about 10 cm, about 15 cm or any other suitable length. In some examples, the needle has a length of between 5 cm-15 cm or about 5 cm-10 cm, or about 10 cm-15 cm or any other suitable range of lengths.

Referring now to FIG. 4., an example needle 218 is shown. The needle 218 has a base 406, and tip 408. The tip 408 is preferably blunt to minimize any damage to the brain from accidental overpenetration. Optionally, a stylet 402 can be removably inserted through the needle lumen 410 and used to puncture through a seal (discussed below) provided at the proximal end of the needle access port for subsequent insertion of the needle through the needle access port. Once the needle is inserted into needle access port of the subdural evacuation port device 100, the stylet can be removed. In some embodiments, the distal end of the needle may be used to puncture the seal directly. A syringe can also be removably attached to the proximal end of the needle (using any now or hereafter known methods) such as after removal of the stylet. For example, FIG. 2 illustrates a syringe 212 attached to the proximal end of a needle (not shown in the figure). The syringe may be used for assisting in the performance of the operation at the blockage site such as, for example, evacuate a blockage through the needle lumen via by creating a suction using the syringe plunger, for delivering a thrombolytic agent (contained within the syringe) to the blockage site, or the like.

Referring back to FIGS. 1-3, the needle access ports 112A, 112B include a depth stop 115 to prevent the tool from entering the tissue beyond a predetermined distance through the distal opening and preventing the tool from entering deeply into the subdural space. In some embodiments, the depth stop 115 is configured to position the needle tip above the distal opening 103A. In other embodiments the depth stop 115 is configured to position the needle tip beneath a blockage or into a blockage and not extend deeply into the subdural space to prevent damage.

In some embodiments, the depth stops 115 can be mounted on a proximal end of each of the needle access ports. In some embodiments, the depth stops 115 are integrally formed with the distal opening 103A of the primary lumen. For example, the distal opening 103 may include a protrusion, tab, or other extension configured as a depth stop. In some embodiments, depth stops 115 are provided or formed within the access port lumens 118 (e.g., as tabs, protrusions, etc.). Other suitable locations of the depth stop within the device 100 are within the scope of this disclosure. Any other types of depth stop that prevents the tool from entering the corresponding access port lumen beyond the predetermined distance are possible. In other examples, the depth stops 115 can be integral with the body (e.g., see FIG. 7).

In various embodiments, the diameter of the depth stop 115 can be configured such that when the needle is inserted into the side port 112A, 112B to a desired length, a stop surface of the needle contacts the depth stop 115 (e.g., see base 406 of needle 218 illustrated in FIG. 3) that prevents further insertion of the needle into the corresponding access port lumen (e.g., from going beyond the subdural space and damaging the brain). In some embodiments, the depth stop 115 contacts a wider portion or stop surface of a needle (e.g., see needle base 406 in FIG. 3). In other embodiments, the depth stop 115 can contact other portions of the needle preventing overpenetration such as a stop surface on the needle or any other suitable location.

The depth stop 115 is configured to be located a distance 123 from the distal opening 103A. The distance 123 can be adjusted by changing the length of the tool being used (while the location of the depth stop is unchanged) and/or by locating the depth stop 115 itself at different locations with respect to the distal opening 103A. In some embodiments, needle access ports having depth stops at different distances from the distal opening can be used to ensure removal of a blockage without penetration of the needle too far into the subdural space. If, for example, a blockage is preventing fluid in the subdural space from being evacuated via the primary lumen 103 (i.e., using the evacuation device 200), a needle can be inserted through one or both of the needle access ports 112A, 112B to perform an operation for clearing the blockage (e.g., dissolve by injection of a thrombolytic agent, dislodge or reposition, break into smaller pieces, or aspirate by suctioning) in order to permit fluid flow to resume without causing damage to the brain. The depth stop 115 may be positioned with respect to the distal opening 103A such that only a predetermined distance 316 of the needle is exposed (e.g., distance 316 in FIGS. 2 and 3). In some embodiments, the predetermined distance can be beneath, above or at the blockage (e.g., the difference between the length of the needle and the distance 123 is the predetermined distance 316).

In some embodiments, the length of the tool is longer than the distance 123 from the depth stop 115 to distal opening 103A of the primary lumen 103 by a predetermined distance 316 such that the tool extends beneath the primary lumen by the distance 316 (see e.g., FIG. 3). In some embodiments, the predetermined distance 316 is about 5 mm, about 10 mm, about 15 mm or any other suitable distance. In some examples, the predetermined distance is between about 5 mm-15 mm, about 7.5-10 mm, about 8 mm-9 mm, or any other suitable range of distances. In some examples, when a blockage is located within the device, the predetermined distance may be such that the tool extends to a distance within the primary lumen 103 and/or is flush with the distal opening 103A of the primary lumen 103. In some examples, the predetermined distance is measured via imaging technology such as computerized tomography to extend beneath or proximate to a blockage prior to insertion of the tool. Other imaging technologies such as ultrasound, magnetic resonance imaging (MRI), or the like are within the scope of this disclosure. In some embodiments the predetermined distance can be measured by viewing the blockage through a transparent portion of the subdural evacuation port device (e.g., a window —provided within the subdural port evacuation device). Such transparent portions of the evacuation port device are described in co-pending U.S. application Ser. No. ______, Attorney Docket Number 349240.01001, filed on Jul. 5, 2023 and having a title “TRANSPARENT SURGICAL EVACUATION PORT DEVICE”, the disclosure of which is incorporated by reference herein in its entirety. Once the predetermined distance is determined, a tool is selected to provide the predetermined distance.

The length of the needle may correlate to the distance 123 from the stop 115 to the distal opening 103A. The distance 123 from the stop 115 to the distal end 106 of body 102 is less than the length of the needle, thus, when inserted only the predetermined distance 316 exits the distal opening 103A. Other suitable predetermined distances 316 that are configured to allow the needle to enter the subdural space 312 without touching brain 310 or other soft tissue are additionally possible. In some embodiments, the subdural evacuation port device 100 can have depth stops 115 located at different lengths to accommodate different length needles.

In some embodiments, a length 127 from distal end 106 of body 102 to the proximal ends of needle access ports 112A, 112B is configured to be such that the distal end of the needle access port(s) lies just above the scalp 302 of the patient when the subdural evacuation port device 100 is mounted onto the skull of a patient. For example, the proximal ends of needle access ports 112A, 112B are about 1 cm about 2 cm, about 2.5 cm, about 3 cm or any other suitable distance from the distal end 106. In some embodiments the proximal ends of needle access ports 112A, 112B are between about 1 cm-3 cm, about 1.5 cm-2.5 cm, about 1.8 cm-2.2 cm, about 1.9 cm-2.1 cm or any other suitable range of distances from the distal end. By configuring the distance in such a manner, the device can avoid widening an incision in a patient's skull when it is mounted onto the skull.

The access port lumens 118 of the needle access ports 112A, 112B have a seal 114 (depicted in the FIGS. as 114A, 114B) at their respective proximal ends. The seal 114 is puncturable by a needle or stylet, but remains sealed such that a suction pressure (e.g., a sub-atmospheric pressure or partial vacuum) is maintained within the primary lumen 103 of the subdural evacuating port device. In some embodiments, the seal is self-sealing. In such embodiments, the seal is made from a self-sealing material that is configured to seal punctures, cracks, or damages to the seal and prevent loss of suction pressure when the needle is inserted into and/or removed from a side port. Accordingly, when the needle is inserted, the seal can close itself around the needle, and when the needle is removed, the seal can close the puncture hole in order to always maintain the suction pressure within the primary lumen. For example, the seal can be a self-sealing stopper or cover placed within and/or over the side port protrusions, similar to those commonly used for vaccine vials, made of rubber or a similar suitable material. As another example, the seal is a cap that fits over a protrusion of the needle access ports, in some such examples, the cap can be held by an adhesive. In other embodiments, the seal is a substantially flat seal held in place by a retainer (e.g., a ring fitting over the seal and the side port protrusion) or adhesive. In some embodiments, the seal can be a collapsible member, such as a sufficiently flexible tube that collapses under a suction is applied by evacuation device and causes sealing of the needle access ports. In other embodiments, the seal is a silicon tip that extends at least partially into the needle access ports.

In some embodiments, a needle access port may form a proximal end of the body 102, while the primary evacuation portion may be provided on the side of the body 102. Specifically, this could permit the needle to be placed straight down through the body 102 and into the blockage.

Referring now to FIG. 2, a system 250 including the subdural evacuation port device 100 of FIG. 1 is shown mounted to a skull 220 of a patient for evacuating excess fluid from the subdural space. FIG. 2 also illustrates the system 250 including an evacuation device 200 that is configured to provide a suction pressure (e.g., a negative pressure, a partial vacuum, or any other force that can cause aspiration of the fluid without causing damage to the brain and/or cause suction of tissue) as the source for withdrawal of fluid through the subdural evacuation port device 100. The evacuation device 200 can be any device that causes aspiration of fluid (e.g., via suctioning, negative pressure, partial vacuum, etc.) through the primary lumen when attached to the proximal end of the primary lumen. The evacuation device 200 may, optionally, be attached to the proximal end of the primary lumen via the fitting 108. Examples of the evacuation device 200 can include, without limitation, a manual evacuation device such as a Jackson-Pratt bulb, an electric pump or other suitable pump or negative pressure device. In certain embodiments, the evacuation device 200 is a manual evacuation device and includes a bulb pump 204. In certain embodiments, a tube 206 can connect the fitting 108 and the bulb pump 204. In certain examples, a tube portion 206, which extends into the bulb pump 204, receives a suction force after the bulb pump 204 is squeezed. In certain embodiments, the bulb pump 204 can include a vent 208 for allowing air to escape the bulb pump 204. In certain examples, the vent 208 is a one-way valve for expelling air from the bulb pump 204 when it is pumped. In some other embodiments, the tube 206 or the bulb pump 204 can include a check valve. The bulb pump 204 can additionally include a plug 210 which can be inserted into vent 208 to prevent fluid from escaping the bulb pump 204. In certain examples, squeezing of bulb pump 204 can create a partial vacuum or suction in the subdural space through the tube 206 and gently aspirate out any fluid in the subdural space through the primary lumen 103 and subsequently tube 202. The tube 202 can be formed from a material that is sufficiently rigid to not collapse under the suction applied by evacuation device 200. In some examples, bulb pump 200 includes a reservoir 205 to contain fluid suctioned evacuation portion. In certain embodiments, the reservoir has a volume about 100 ml, about 125 ml, about 150 ml, about 175 ml or any other suitable. In some other examples, the reservoir 205 has a volume of about 100 ml-175 ml, about 120 ml-160 ml, 130 ml-150 ml, 135 ml-145 ml or any other suitable range of volumes.

In various embodiments, the magnitude of the suction pressure exerted by a typical evacuation device is about 0.8 inch to 1 inch of mercury (Hg) with respect to atmospheric pressure. Depending on the procedure, it will be appreciated that a lower level (e.g., less than 0.8 inches of mercury) of suction pressure may be used. While relatively higher levels of suction pressure may be used (such as up to approximately 1.2 inches of mercury), significantly higher levels of suction pressure can damage brain tissue and/or hamper the recovery of the brain and the associated tissues, by, for example, causing hemorrhages to occur. As such, an optimal suction pressure may be used that permits the suction pressure condition to be maintained in the subdural space of the patient for a relatively extended period of time for removing any further collection of fluid, as well as promoting a gradual expansion of the brain in the subdural space during the healing process. The components used to construct subdural evacuation port device are sufficiently strong to sustain a suction of this level without collapsing (discussed in further detail below). As discussed above, it will be appreciated that any suitable evacuation device providing such pressure can be configured to attach to the primary evacuation portion 105. In some examples, the negative pressure is about 0.5 in of Hg about 0.6 in of Hg, about 0.7 in of Hg. 0.8 in/Hg, about 0.9 in of Hg, about 1 in of Hg, or any other suitable negative pressure. In some embodiments, the negative pressure is between about 0.5 in of Hg-1 in of Hg, about 0.55 in of Hg 0.95 in of Hg, about 0.7 in of Hg-0.9 in of Hg, about 0.75 in of Hg-0.85 in of Hg or any other suitable ranges of pressure.

FIG. 3 also illustrates an evacuation device (as discussed above) configured for evacuating fluid through the opening 105A of the evacuation portion 105. An example blockage 314 is shown to be located at the distal opening 103A that may slow or stop fluid entry during fluid evacuation, via the primary lumen 103 when a suction pressure is applied. A needle 218 having a needle lumen 410 is insertable into one of access port lumens 118 through the seal 114A, 114B to access the site of the blockage 314, and subsequently perform an operation to remove, dislodge, dissolve (e.g., by introducing a thrombolytic agent), and/or break the blockage 314. In other embodiments, the needle is configured to extend into the blockage from a proximal side. As depicted, the needle 218 extends into the subdural space by the predetermined distance 316, that is controlled using the depth stop 115. The blockage, the dissolved debris of the blockage and/or partially broken down pieces of the blockage may be aspirated via the needle and/or via the primary lumen itself.

A withdrawal mechanism can then be attached to the needle to remove the blockage 314 (or debris/portions thereof). In some embodiments, the withdrawal mechanism is directly attached to the needle. In some embodiments, the withdrawal mechanism is a syringe 212 that is connected to needle 218 for suctioning the blockage. In some embodiments, a suction force is applied through the needle lumen 410 thereby removing the blockage. The suction force can be applied using a plunger 214. Other withdrawal mechanisms are additionally possible such as an electric withdrawal mechanism that provides suction to the needle lumen.

Additionally and/or alternatively (discussed in further detail below), the needle is configured to provide a thrombolytic agent to the blockage 314. For example, the syringe 212 can be filled with a thrombolytic agent which can be introduced to the blockage by depressing the plunger 214 and evacuating the thrombolytic agent into the blockage. The thrombolytic agent may at least partially dissolve the blockage. The syringe may then, optionally, be used to suction the debris of the thrombolized blockage and/or any non-thrombolized portions of the blockage. Optionally, the debris of the thrombolized blockage and/or any non-thrombolized portions of the blockage may be aspirated via the primary lumen along with the subdural fluid. Thrombolytics are medications used to dissolve blood clots, specifically thrombi by activating the fibrinolytic system. Examples of the thrombolytic agent can include, without limitation, issue plasminogen activator (tPA), streptokinase (SK), and urokinase (UK), Alteplase, Reteplase, Tenecteplase, streptokinase or any other suitable thrombolytic agent.

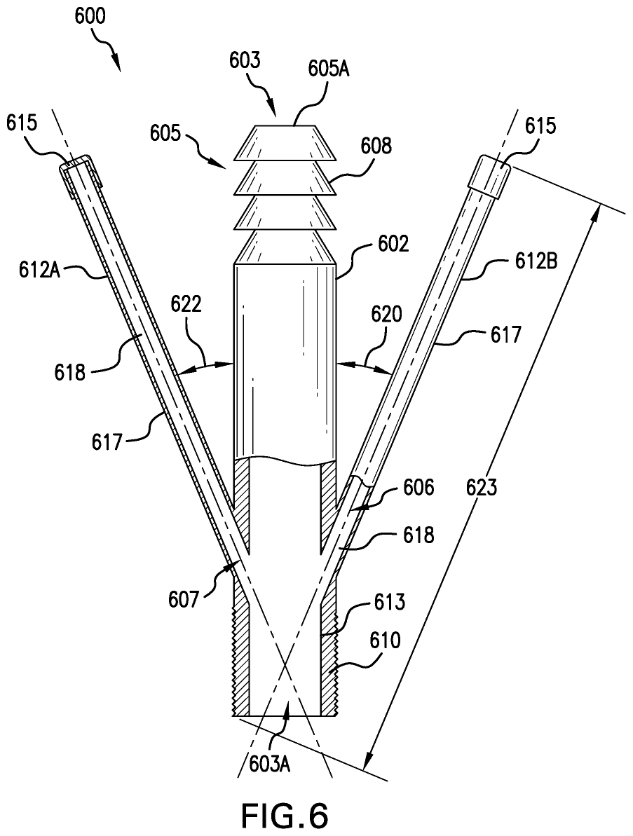

Referring to FIGS. 6, a different embodiment of a subdural evacuation port device is described. Referring to the embodiment of FIG. 6, a subdural evacuation port device 600 has two needle access ports 612A, 612B defined by protrusions and access port lumens 618 (similar to the needle access ports 112A, 112B). The subdural evacuation port device includes a primary lumen 603 (similar to the primary lumen 103) and includes an evacuation portion 605 having a primary evacuation opening 605A. The needle access ports 612A, 612B are angled at angles 622, 620 with respect to the lumen 603, respectively, the evacuation port device has a fitting 608 on the evacuation portion 605 and a skull engagement region 610 that are similar to the fitting 108 and the skull engagement region 110. The protrusions 612A, 612B include stops 615 similar to the depth stops 115. The angles can be the same or different and are configured such that half of the diameter of a corresponding needle that inserted along centerlines 606, 607 is less than the distance from one of the centerlines 606, 607 to the inner wall 613 of body 602. In other words, the centerlines 606, 607 of the needle access ports intersect a distal opening 603A of the lumen 603. This configuration can be useful if a blockage occurs below the distal opening 103A such that a needle can be inserted to break up the blockage. The protrusions 612A, 612B include stops 615 and are located at a distance 623 (similar to the distance 123) from the distal end of the device. The stops 615 include a seal similar to seal 114.

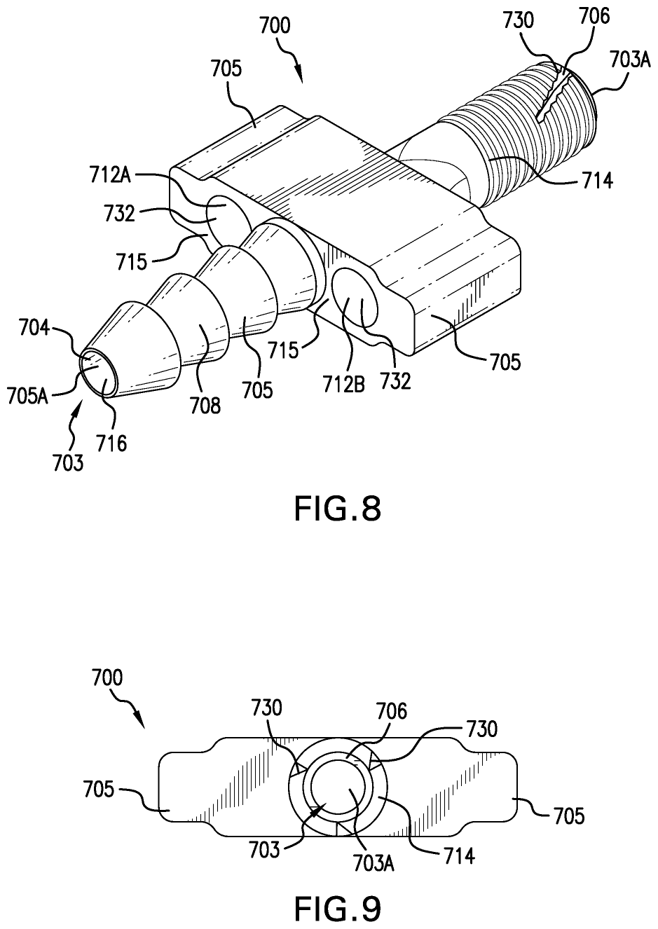

Referring to FIGS. 7-9, a different embodiment of a subdural evacuation port device 700 having unitary body is described. The subdural evacuation port device 700 is similar to the subdural evacuation port device 100 and 600, with some differences including self-tapping into a hole (such as the hole 330 shown in FIG. 3) that has been drilled into a skull. The subdural evacuation port device 700 includes a body 710 which includes a primary lumen 703 that extends from a distal end 706 to a proximal end 704. The lumen 703 includes a distal opening 703A. The subdural evacuation port device 700 includes an evacuation portion 705 with a primary evacuation opening 705A. The evacuation portion 705 includes a fitting 708 attachable to an evacuation device at the proximal end and has distal portion that is contiguous with a skull engagement region 714 of the subdural evacuation port device 700.

The body 710 additionally includes a first and a second needle access ports 712A, 712B similar to the needle access ports 112A, 112B. The needle access ports include a depth stop 715 at a distance 723 (similar to distance 123) from the distal opening 703A which is formed with the body 710. The first and second ports 712A, 712B each define access port lumens 718 that extend through the body 710. The first and second ports 712A, 712B extend through the body 710 at angles 720, 722 relative to the lumen 703. The angles 720, 722 are similar to the angles 120, 122. The access port lumens 718 of the first and second needle access ports 712A, 712B have a non-uniform diameter lumen that changes from a minimum diameter (that is sufficiently large to accommodate a needle) to a maximum diameter in the proximal to distal direction, where the maximum diameter is configured to receive a plug 732 which can be similar to the seal 114. The first and second needle access ports 712a, 712b can be drilled directly into the body 710 using a suitable drill having a similar diameter to the desired diameters of the needle access ports 712A, 712B. The nonuniform lumen changes in a stepwise manner in some examples. In some examples, the maximum diameter has a taper to guide the needle to the minimum diameter. In some examples, the maximum diameter is in the proximal direction relative to the body 710 and the minimum diameter extends from the end of the maximum diameter to the lumen 703. Other configurations are additionally possible. The body is located at a length 727 from the distal end 706 similar to the length 127.

The skull engagement region 714 of the subdural evacuation port device 700 extends from the body 710 to the distal end 706. The lumen 703 extends through the skull engagement region 714 to the proximal end 704. The skull engagement region 714 includes pipe threading as discussed above. In some examples, the skull engagement region 714 includes flutes 730 for tapping into the hole created by a drill as discussed above. In some examples, the skull engagement region 714 includes three flutes 730, however, more or less flutes can be included depending on the desired use.

Optionally, the body 710 includes wings 705 that are integral with the body 710. In some embodiments, the body 710 and wings 705 are rigid. The wings 705 facilitate rotation of the subdural evacuation port device 700. The wings 705 extend in opposite directions from the body 710 with respect to one another. The first and second ports 712A, 712B are adjacent to the wings 705. The body 710 and the wings 705 are sufficiently rigid to allow for rotation and threading of a hole (e.g., the hole 330).

As disclosed herein the body 102, 602, 702 can be made of suitably rigid materials, such as metals, for example, titanium, steel, stiff plastics, or any other suitable material. In some embodiments, the needle access ports, when configured as protrusions (e.g., 112A, 112B, 612A, 612B) can be made from a different material from the body. In some embodiments, the subdural evacuation port device 100, 600, 700 is made from a material that is sufficiently rigid to be screwed into a skull of a human. In some examples, the subdural evacuation port device 100, 600, 700 is formed from a material that is sufficiently rigid to not collapse when subjected to suction pressure applied for removal of fluid and or the blockage.

In some embodiments, the subdural evacuation port device 100, 600, 700 is made from a material having a substantially low thrombogenicity (e.g., thrombogenicity means the tendency to of material in contact with blood to form a thrombus or clot), such that it does not cause blood clotting or minimizes blood clotting. In some embodiments, the material is substantially less thrombogenic than stainless steel. In some examples, the material is half as thrombogenic as stainless steel. In other examples, the material is less than half as thrombogenic as stainless steel. Other suitable embodiments are additionally possible.

In some embodiments, the subdural evacuation port device 100, 600, 700 is made from a material that is transparent or partially transparent with respect to an imaging modality being used such that an operator can visualize the interior of the device (e.g., a needle advanced within the device may be visible) when the device is mounted within a skull during operation and is viewed using the corresponding imaging device. For example, the device 100, 600, 700 (or portions thereof) can be made from a material that is transparent or only partially transparent when viewed using the imaging device for imaging modalities, such as computerized tomography (e.g., CT scan), MRI (magnetic resonance imaging), a camera, or X-ray or other suitable imaging technologies. In some examples, the device is made from a radiolucent material (i.e., a material has that allows for x-ray beams to pass through the material, radio dense materials do not allow for passage of radio waves) that is transparent or partially transparent using CT scans or X-rays. Examples include, without limitation, plastics, thermoplastics, most polymers (e.g., polymers that are not radio-dense), polycarbonate, polyacetal. In some examples, the device is made from a material that is not visible or only partially visible to an MRI imaging device such as, without limitation, ceramic, plastic, polymers, polyacetal, polycarbonate. In some embodiments, the device 100, 600, 700 is made from a non-magnetic material that is compatible with an MRI machine because magnetic materials can move and heat up when used in MRI machines which can create danger to the patient by moving the device or heating up the device. As such, when a tool (such as a needle) is inserted through, for example, the needle access ports, it can be viewed without any obstructions caused by the device and without the device moving. In some other embodiments, the device 100, 600, 700 (or portions thereof) can be formed from a material that is both transparent when using an imaging device and has a low thrombogenicity. In some examples, the device (or portions thereof) can be formed from a material that is transparent when using an imaging device, has a low thrombogenicity, and is sufficiently rigid as discussed above.

In some embodiments, the device 100, 600, 700 is made from a polymer. One suitable polymer is polyacetal. Polyacetal is a formaldehyde-based, semi-crystalline thermoplastic. Polyacetal is substantially less thrombogenic than metals. Polyacetal is radiolucent and is not magnetic be used with imaging technologies such as CT scanning and MRI without causing interference or other issues. Additionally, Polyacetal is recyclable after use. Other suitable polymers such as acrylonitrile butadiene styrene (ABS), polycarbonate, acetate or any other suitable materials or polymers are additionally possible.

In other embodiments, the subdural evacuation port device can include one or more components (e.g., lumen, body, port, a window, etc.) and/or substantially all that is optically transparent, meaning light is allowed to pass through with minimal absorption. Such transparent subdural port devices are described in U.S. application Ser. No. ______, Attorney Docket Number 349240.01001, filed Jul. 5, 2023 and having a title “TRANSPARENT SURGICAL EVACUATION PORT DEVICE”, the disclosure of which is incorporated herein in its entirety. In some embodiments, the optically transparent material may be, without limitation, crystalized ABS, polycarbonate, glass, acrylic, or a transparent ceramic such as aluminum oxynitride or any other medical grade, shatter resistant materials. In some embodiments, the subdural evacuation port can be formed from a material that has a substantially low thrombogenicity, is optically transparent and sufficiently rigid to sustain a suction or any combination of properties as discussed above.

Disclosed embodiments relate to a kit for evacuating fluid from the subdural space of the skull including a subdural evacuation port device 100 or 600 or 700 and a needle 218. The needle can be a lumbar puncture needle. In some embodiments, the kit can further include at least one of a stylet or a syringe. In certain embodiments, the kit can additionally include an evacuation device such as a bulb pump 204. Disclosed embodiments also include a kit for evacuating fluid from the subdural space of the skull including a subdural evacuation port device 100 or 600 and a tap. Disclosed embodiments also include a kit for evacuating fluid from the subdural space of the skull including a subdural evacuation port device 100 or 600 and a surgical drill. It will be appreciated that the kit can include any or all of the devices discussed above as well as any other suitable devices required for proper evacuation of subdural space of a patient.

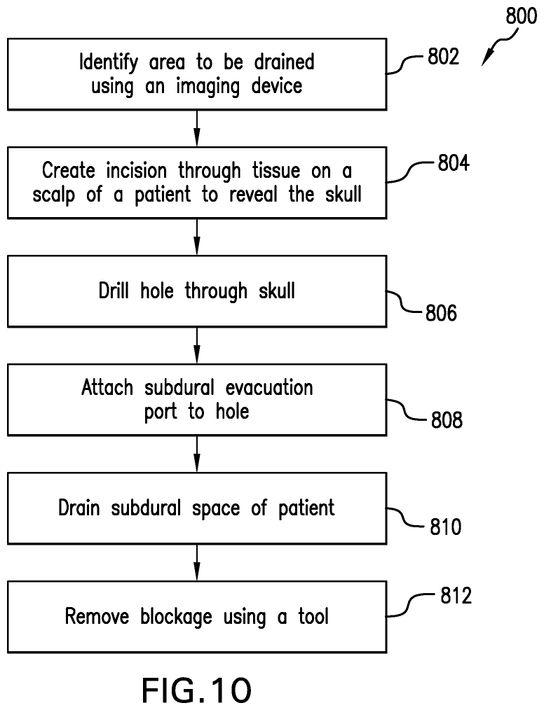

Referring to FIG. 10, a flowchart 800 describing an embodiment of a method draining subdural space of a patient using a device such as the devices 100, 600, 700 is discussed. Prior to the steps provided by the flowchart, anesthesia such as local or general anesthesia can be given to the patient. The flowchart includes a step 802 of identifying a location desired to be drained on a patient. In some examples, the location is identified using imaging technology (e.g., CT, X-ray, or MRI). In some embodiments, the location is an area on the patient with the greatest subdural hematoma thickness.

A step 804 includes creating an incision on the scalp of a patient. In some embodiments, the incision can be made through skin, subcutaneous tissue, galea, and periosteum. In some examples, a relatively small incision is made on the scalp of a patient which allows for a drill bit to drill a hole into the skull.

In a step 806 a hole is drilled at the location of the incision. The hole can be drilled using a suitable surgical drill. A hole (e.g., hole 330 of FIG. 3) can then be drilled through the skull into the cranial cavity (e.g., dura 308 or subdural space 312 of FIG. 3) using a suitable surgical or cranial drill at the location of the incision. In some examples, the drill is adjusted based on the imaging provided by step 802 such that the drill does not drill beyond the desired depth allowing the drill to advance until the drill reaches the space that is desired to be drained and can be withdrawn removing any bone fragments from the skull. The drill bit holds the edges of the incision apart. After the hole is drilled, the subdural evacuation port device can be secured within the hole, at least partially. Optionally, the hole can be tapped with threads by a tap having a similar diameter to the hole. For example, the tap can be a taper tap, a bottoming tap, a plug tap or a different suitable tap. In some examples, the tap has a similar threading to the threads of the skull engagement region 110. Once a threading that is similar to the threading of a subdural evacuation port device has been made, the subdural evacuation port can be screwed within the hole.

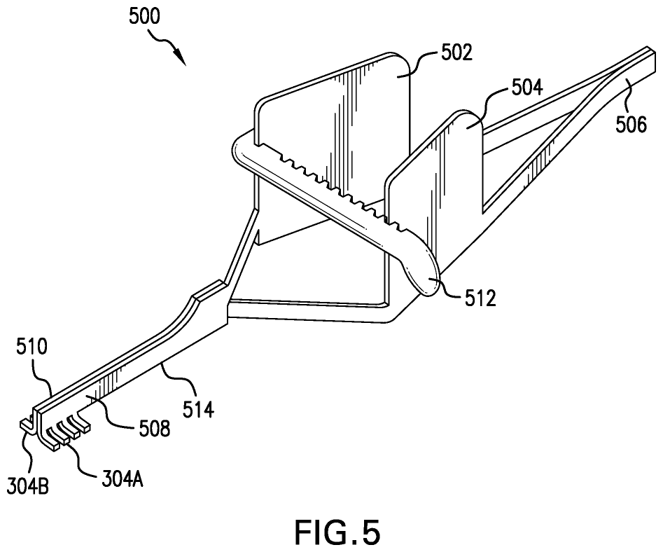

Optionally, a retractor can then be provided at the incision such as the retractor 500. Referring to FIG. 5, in some embodiments a retractor 500 is used to hold the incision 302A apart. The retractor 500 can the form of a “Holzheimer” retractor. The “Holzheimer” retractor generally has two arms 502, 504 that are joined together at proximal ends of the arms to form an apex 506. The arms 502, 504 extend away from the apex 506 and terminate at free ends 508, 510 of the arms. Preferably, the free ends of the arms are spaced such that the arms form a substantially V-shaped structure. A locking member 512 may be included on the retractor for selectively locking the arms at a desired spacing. The “Holzheimer” retractor 500 has a lower edge 514 for inserting into the incision. Tabs 304A, 304B may be provided on each of the arms 502, 504 adjacent to the lower edge 514 at a location separated from the apex 506 of the clip. The tabs 304A, 304B preferably lodge themselves below the outer surface of the scalp to help hold the clip in place with respect to the incision during the period when the incision needs to be held open. Optionally, retraction of the scalp may be performed by other known types of surgical retractors, such as, for example, a “Mastoid” retractor, a “Gelpi” retractor, or a “Heiss” retractor.

At a step 808 a subdural evacuation port device such as the subdural evacuation port device 100, 600, 700 can mounted and secured into the hole using the threads. It will be appreciated that if the device is self-tapping such as the device 700, using a tap may not be necessary and steps 808 and 810 can be combined. For example, the dura can be punctured by a needle, stylet, or other device, and the evacuation port device may be mounted at the puncture site via the self tapping threads.

In a step 810, the subdural space of the patient can be drained using the methods provided herein. If a blockage occurs at the drainage location, step 812 provides clearing the blockage. The blockage can be removed by performing an operation at the site of blockage using a tool (such as the needle 218) through a side port of a device 100, 600, 700 as discussed herein. The operator can view the blockage using imaging technology, as discussed herein, and select a needle having an appropriate length. In some examples, the operator can introduce a thrombolytic agent, as discussed above, to assist with dissolving the blood clot. In other examples, the operator can withdraw the blockage, blockage debris, partially dissolved blockage, etc. via the needle (and/or the primary lumen). The operator can additionally withdraw the blockage or clot if it is determined that the thrombolytic agent is not effective and position the needle effectively for withdrawal It will be appreciated the steps outlined above include other steps discussed throughout the present disclosure and are for explanatory purposes, for example penetrating the dura of the patient prior to draining the hole. Once, the drainage is complete, the device can be left within the skull and another image can be taken using imaging technology to determine if the fluid has been completely evacuated from the subdural layer of the brain. The device can then be removed once evacuation has completed.

Other advantages of the present invention can be apparent to those skilled in the art from the foregoing specification. Accordingly, it will be recognized by those skilled in the art that changes, or modifications may be made to the above-described embodiments without departing from the broad inventive concepts of the invention. It should therefore be understood that this invention is not limited to the particular embodiments described in this document but is intended to include all changes and modifications that are within the scope and spirit of the invention as defined in the claims.

The term “approximately,” when used in connection with a numeric value, is intended to include values that are close to, but not exactly, the number. For example, in some embodiments, the term “approximately” may include values that are within +/−5 percent of the value.

This disclosure is not limited to the particular systems, methodologies or protocols described, as these may vary. The terminology used in this description is for the purpose of describing the particular versions or embodiments only and is not intended to limit the scope. It will be understood that terms such as “same,” or “equal,” as used herein when referring to orientation, layout, location, shapes, sizes, amounts, or other measures do not necessarily mean an exactly identical orientation, layout, location, shape, size, amount, or other measure, but are intended to encompass nearly identical orientation, layout, location, shapes, sizes, amounts, or other measures within acceptable variations that may occur, for example, due to manufacturing processes. The term “substantially” may be used herein to emphasize this meaning, unless the context or other statements clearly indicate otherwise. For example, items described as “substantially the same,” “substantially equal,” or “substantially planar,” may be exactly the same, or equal, or may be the same, equal, or planar within acceptable variations that may occur, for example, due to manufacturing processes and/or tolerances. The term “substantially” may be used to encompass this meaning, especially when such variations do not materially alter functionality. As used herein, the term “proximal” means closest to the operator (less into the body) and “distal” means furthest from the operator (further into the body). In positioning a medical device from a downstream access point, distal is more upstream and proximal is more downstream.

It will be understood that various modifications may be made to the embodiments disclosed herein. Likewise, the above disclosed methods may be performed according to an alternate sequence. Therefore, the above description should not be construed as limiting, but merely as exemplification of the various embodiments. Those skilled in the art will envision other modifications within the scope and spirit of the claims appended hereto.

What is claimed is:

1. A subdural evacuation port device, comprising a body that includes:
a distal opening at a distal end,
a primary evacuation opening at a proximal end;
a skull engagement region that at least partially surrounds the distal end of the body, and that is configured to engage with a skull of a patient; and
a primary lumen extending from the distal opening to the primary evacuation opening,
wherein the body is formed from a polymer that is sufficiently rigid to sustain a suction in the lumen to allow withdrawal of subdural fluid from the distal opening through the lumen to a suction device connected to the primary evacuation opening.

2. The subdural evacuation port device of claim 1, wherein the polymer has a substantially low thrombogenicity.

3. The subdural evacuation port device of claim 1, wherein the subdural evacuation port device is compatible for use during computerized tomography (CT) scans.

4. The subdural evacuation port device of claim 1, wherein the polymer is polyacetal.

5. The subdural evacuation port device of claim 1, further comprising:
a needle access port defining an access port lumen in communication with the primary lumen; and
a seal at a proximal end of the access port lumen that seals the access port lumen to preserve the suction within the lumen and that is penetrable by a needle.

6. The subdural evacuation port device of claim 5, wherein the needle access port is disposed and oriented with respect to the body to allow a needle inserted into the access port lumen to extend into the primary lumen.

7. The subdural evacuation port device of claim 1, wherein the polymer is optically transparent.

8. The subdural evacuation port device of claim 1, wherein the skull engagement region comprises threads for cutting threads into the skull of the patient.

9. The subdural evacuation port device of claim 8, wherein the threads are fluted and self-tapping.

10. A subdural evacuation port device, comprising:
a body that includes:
a distal opening,
a primary evacuation opening, and
a primary lumen extending from the distal opening to the primary evacuation opening; and
a needle access port defining an access port lumen that is in communication with the primary lumen, wherein the needle access port is positioned with respect to the body to direct a needle inserted within the access port lumen into the primary lumen.

11. The subdural evacuation port device of claim 10, wherein the needle access port includes a stop surface positioned at a first distance away from the distal opening to stop insertion of the needle past a threshold depth of insertion into the access port lumen.

12. The subdural evacuation port device of claim 10, wherein the primary evacuation opening is configured to connect to an evacuation device that is configured to provide suction through the primary lumen.

13. The subdural evacuation port device of claim 10, further comprising a seal that seals the access port lumen to preserve suction within the primary lumen and that is penetrable by the needle.

14. The subdural evacuation port device of claim 10, further comprising a second needle access port defining a second access port lumen in communication with the primary lumen, wherein the second needle access port is configured direct a needle inserted within the second access port lumen into the primary lumen and at a location or an orientation that is different from that of a needle inserted within the access port lumen.

15. The subdural evacuation port device of claim 10, wherein the subdural evacuation port device is compatible for use during computerized tomography (CT) scans.

16. The subdural evacuation port device of claim 10, wherein the body is formed from a polymer.

17. The subdural evacuation port device of claim 10, wherein the subdural evacuation port device is formed from an optically transparent material.

18. A kit for evacuating fluid from subdural space of a skull, comprising:
the subdural evacuation port device of claim 10; and
a needle configured for insertion into the port lumen for removal of a blockage within the primary lumen.

19. The kit of claim 18, wherein the needle access port of the subdural evacuation port device includes a stop surface positioned at a first distance away from the distal opening to stop insertion of the needle past a threshold depth of insertion into the access port lumen.

20. The kit of claim 18, wherein the needle is a lumbar puncture needle.

21. The kit of claim 18, further comprising an evacuation device that is configured to provide suction through the primary lumen.

22. The kit of claim 21, wherein the evacuation device is a bulb pump.

23. The kit of claim 18, wherein the needle is hollow and includes a needle lumen.

24. The kit of claim 23, further comprising a stylet configured to removably extend through the needle lumen to puncture a seal that seals the access port lumen of the subdural evacuation port device while preserving suction within the primary lumen.

25. A method of using the subdural evacuation port device of claim 10, comprising the steps of:
drilling a hole through a skull and dura of a patient;
mounting the subdural evacuation port device to the hole via a skull engagement region surrounding a distal end of the body;
applying suction within the primary lumen to withdraw subdural fluid from the dura of the patient through the primary lumen;
determining whether there is a blockage preventing subdural fluid from flowing through the primary lumen; and
in response to determining that a blockage is preventing subdural fluid from flowing through the primary lumen, removing the blockage.

26. The method of claim 25, further comprising determining the cause of the blockage by performing a CT scan.

27. The method of claim 25, further comprising removing the blockage by inserting a hollow needle through the access port lumen into the primary lumen.

28. The method of claim 27, further comprising injecting a thrombolytic agent via a hollow needle lumen of the needle.

29. The method of claim 27, further comprising suctioning the blockage using the needle.
